(19) United States
(12) Patent Application Publication
(10) Pub. No.: US 2002/0042050 A1
(43) Pub. Date: Apr. 11, 2002

(54) METHODS FOR DIAGNOSING GLAUCOMA AND DISCOVERING ANTI-GLAUCOMA DRUGS

(76) Inventors: ABBOT F. CLARK, ARLINGTON, TX (US); ROBERT J. WORDINGER, EULESS, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/308,295

(22) PCT Filed: Nov. 14, 1997
(86) PCT No.: PCT/US97/21054

(51) Int. Cl. C12Q 1/68

(52) U.S. Cl. 435/6

(57) ABSTRACT

Methods for diagnosing glaucoma and for screening therapeutic agents for their usefulness in treating glaucoma based on the detection of aberrant expression of beta glucocorticoid receptor (GRbeta).

Priority is claimed from the provisional application, U.S. patent application Ser. No. 60/033227 filed Dec. 5, 1996.

### BACKGROUND OF THE INVENTION

Glaucoma is usually diagnosed by monitoring a patient's visual field loss, changes in the appearance of their optic disc, and their intraocular pressure. Glaucoma is currently treated using one or more of three strategies to lower the elevated intraocular pressure associated with the disease: with pharmaceuticals (such as beta-blockers, carbonic anhydrase inhibitors, and miotics), with laser trabeculoplasty, and/or with glaucoma filtration surgery. All of these therapies indirectly lower intraocular pressure but do not address the underlying disease process occurring in the trabecular meshwork. It would be advantageous to be able to diagnose glaucoma before a patient begins experiencing a loss in their visual field and deterioration of their optic disc.

There is a large body of evidence suggesting that glucocorticoids are involved in the generation of ocular hypertension and glaucoma. See Clark, A. F., Journal of Glaucoma, “Steroids, Ocular Hypertension, and Glaucoma,” 4:354-369, 1995. Several investigators have shown that the human trabecular meshwork (TM) contains the classical glucocorticoid receptor (GRα). See Weinreb, et al., Invest. Ophthalmol. Vis. Sci., “Detection of Glucocorticoid Receptors in Cultured Human Trabecular Cells,” 21:3, 403407, 1981, and Hernandez, et al., Invest. Ophthalmol. Vis. Sci., “Glucocorticoid Target Cells in Human Outflow Pathway: Autopsy and Surgical Specimens,” 24:1612-1616, 1983. Recently, the expression of an alternatively spliced form of the human glucocorticoid receptor (GRβ) was discovered in non-ocular tissues and cells. See Bamberger, et al., The Journal of Clinical Investigation, “Glucocorticoid Receptor β, a Potential Endogenous Inhibitor of Glucocorticoid Action in Humans,” 95:2435-2441, 1995, and Oakley, et al., The Journal of Biological Chemistry, “The Human Glucocorticoid Receptor β Isoform,” 271:16, 9550-9559, 1996. This alternatively spliced form of the glucocorticoid receptor (GR) is expressed as a protein which no longer binds glucocorticoids, but is able to interfere with the activated form of the normal glucocorticoid receptor and block or alter physiological functions of the glucocorticoid receptor.

WO 96/14411 discloses a method for diagnosing glaucoma in a patient which comprises determining whether the amount of a trabecular meshwork induced glucocorticoid response protein present in the trabecular meshwork of an eye of a patient exceeds the amount of that trabecular meshwork induced glucocorticoid response protein present in the trabecular meshwork of an eye of an individual who is not suffering from glaucoma, wherein the detection of an excessive amount of the trabecular meshwork induced glucocorticoid response protein is indicative of Glaucoma.

### SUMMARY OF THE INVENTION

The present invention is directed to methods for diagnosing glaucoma by testing a person for aberrant GRβ expression. Also set forth are methods for screening for therapeutic agents useful for treating glaucoma.

### DESCRIPTION OF PREFERRED EMBODIMENTS

Surprisingly, it has been found that cultured human trabecular meshwork cell lines derived from glaucomatous donors express mRNA for both an alternate splice form of the human glucocorticoid receptor (GRβ), as well as the normal glucocorticoid receptor (GRα), whereas normal TM cell lines only express mRNA for GRα. It is believed that the elevated intraocular pressure associated with primary open-angle glaucoma may be due to the aberrant expression of GRβ in the trabecular meshwork. Therefore, determining that an individual abnormally expresses GRβ in their trabecular meshwork or other tissues can lead to a diagnosis of glaucoma. Also, this discovery can be used to determine whether agents have therapeutic value in treating glaucoma by determining whether they interact with GRβ or alter the expression of GRβ. This can be done using ligand binding assays or GRβ functional assays.

Diagnosing aberrant GRβ expression or defects in the GR gene which encodes GRβ can be done by using procedures well known to those skilled in the art. See Caskey, C. T., J.A.M.A., “Molecular Medicine. A Spin-off From the Helix,” 269:15, 1986-1992, 1993. For example, subjects could be screened for the presence of a genetic defect in GRβ by analyzing the DNA derived from peripheral blood leukocytes. Types of DNA analyses could include, but would not be limited to: restriction fragment length polymorphisms (RFLP), single-stranded conformation polymorphisms (SSCP), polymerase chain reaction (PCR), denaturing gradient gels, allele specific oligonucleotide ligation assay, and allele specific hybridization assay. In addition, trabecular meshwork, or other relevant cells from subjects could be analyzed for GRβ expression by a number of techniques such as reverse-transcription polymerase chain reaction (RT-PCR), immunoassays, GR functional assays, etc.

We claim:

1. A method for diagnosing glaucoma which comprises detecting aberrant alternate splice form of the human glucocorticoid receptor (GRβ) expression or defects in a GR gene which encodes GRβ.

2. The method of claim 1 wherein GR gene defects are detected by a method selected from the group of assays consisting of: restriction fragment length polymorphism (RFLP), single-stranded conformation polymorphism (SSCP), polymerase chain reaction (PCR), denaturing gradient gel, allele specific oligonucleotide ligation, and allele specific hybridization.

3. A method for diagnosing glaucoma, which comprises detecting genetic changes in the GR gene leading to altered GRβ expression.

4. A method for diagnosing glaucoma, which comprises detecting genetic changes outside the GR gene which lead to altered GRβ expression.

5. A method for determining whether an agent is useful for treating glaucoma by determining whether it interacts with GRβ or alters the expression of GRβ.
